(19) United States
(12) Patent Application Publication
(10) Pub. No.: US 2014/0113364 A1
(43) Pub. Date: Apr. 24, 2014

(54) CELL CULTURE DEVICE

(71) Applicant: SAMSUNG ELECTRO-MECHANICS CO., LTD., Suwon (KR)

(72) Inventors: Sang Jin KIM, Suwon (KR); Dong Woo LEE, Suwon (KR); Bo Sung KU, Suwon (KR)

(73) Assignee: SAMSUNG ELECTRO-MECHANICS CO., LTD., Suwon (KR)

(21) Appl. No.: 13/735,483

(22) Filed: Jan. 7, 2013

(30) Foreign Application Priority Data
Oct. 18, 2012 (KR) 10-2012-0116168

(51) Int. Cl. C12Q 1/02

(52) U.S. Cl. CPC C12Q 1/025
USPC 435/297.1; 435/303.1; 435/289.1

(57) ABSTRACT

There is provided a cell culture device including: a cell chip receiving part receiving a cell chip; a drug storing part storing a drug; and a circulation part connecting the cell chip receiving part and the drug storing part to one another and circulating the drug therebetween. The cell culture device may allow for the observation or examination of a reaction between a cell and a drug in an environment similar to that of the inside of a body.

### CROSS-REFERENCE TO RELATED APPLICATIONS

This application claims the priority of Korean Patent Application No. 10-2012-0116168 filed on Oct. 18, 2012, in the Korean Intellectual Property Office, the disclosure of which is incorporated herein by reference.

### BACKGROUND OF THE INVENTION

1. Field of the Invention

The present invention relates to a cell culture device, and more particularly, to a cell culture device capable of examining a reaction between a cell and a drug in an environment similar to that of the inside of a body.

2. Description of the Related Art

The demand for biomedical apparatuses and biotechnology for rapidly diagnosing various human diseases has recently increased. Therefore, the development of experimental apparatuses and tools capable of rapidly providing diagnostic results for specific diseases, diagnoses of which, previously conducted in a hospital or a research laboratory, have required a relatively long period of time, has been actively conducted.

Meanwhile, in order to develop new drugs and examine the stability thereof, it is necessary to observe a reaction between new drugs (that is, medicines) and cells. In general, a reaction experiment between drugs and cells is conducted by using a culture dish, or the like.

However, since a reaction between a drug and a cell conducted in a culture dish is significantly different from a reaction between a drug and a cell occurring inside a body, it is difficult to accurately observe or examine the reaction between a drug and a cell with only reference to experimental results from experiments performed in culture dishes. Therefore, the development of a new device capable of observing reactions between drugs and cells in an environment similar to the inside of a body is required.

Meanwhile, as related art documents searched by the applicant, there are provided patent documents 1 and 2. However, neither of these patent documents, which relate to a polymerase chain reaction (PCR), are appropriate for solving the above-mentioned problems.

### RELATED ART DOCUMENT

(Patent Document 1) KR2005-117811 A
(Patent Document 2) JP2010-203779 A

### SUMMARY OF THE INVENTION

An aspect of the present invention provides a cell culture device capable of observing or examining a reaction between a cell and a drug in an environment similar to that inside a body.

According to an aspect of the present invention, there is provided a cell culture device including: a cell chip receiving part receiving a cell chip; a drug storing part storing a drug; and a circulation part connecting the cell chip receiving part and the drug storing part to one another and circulating the drug therebetween.

The cell chip receiving part may have a passage determining a path for movement of the drug.

The passage may have a zigzag shape.

The drug storing part may be divided into a plurality of drug storing spaces so as to individually store the same or different types of drug.

The circulation part may be coupled to and separated from the cell chip receiving part and the drug storing part so as to selectively connect the plurality of drug storing spaces and the cell chip receiving part to one another.

The circulation part may include: a pair of pipes connecting the drug storing part and the cell chip receiving part to one another; and a pump installed on the pipe.

At least one of the cell chip receiving part and the drug storing part may include a filter for filtering a foreign material included in the drug circulating between the cell chip receiving part and the drug storing part.

According to another aspect of the present invention, there is provided a cell culture device including: a lower body having a receiving space; a cell chip receiving part mounted in the receiving space and receiving a cell chip; a drug storing part mounted in the receiving space and storing a drug; a circulation part connecting the cell chip receiving part and the drug storing part to one another and circulating the drug therebetween; and an upper body coupled to the lower body to close the receiving space.

The cell chip receiving part may have a passage determining a path for movement of the drug.

The passage may have a zigzag shape.

The drug storing part may be divided into a plurality of drug storing spaces so as to individually store the same or different types of drug.

The circulation part may be coupled to and separated from the cell chip receiving part and the drug storing part so as to selectively connect the plurality of drug storing spaces and the cell chip receiving part to one another.

The circulation part may include: a pair of pipes connecting the drug storing part and the cell chip receiving part to one another; and a pump installed on the pipe.

At least one of the cell chip receiving part and the drug storing part may include a filter for filtering a foreign material included in the drug circulating between the cell chip receiving part and the drug storing part.

The cell culture device may further include a humidity control part formed in the receiving space to control humidity therein.

The humidity control part may include a water storing space storing water, and a heater heating the water in the water storing space.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a view showing a cell culture device according to an embodiment of the present invention;

FIGS. 2 through 4 are plan views showing different forms of a cell chip receiving part shown in FIG. 1;

FIG. 5 is a view showing a state in which a cell chip is mounted in the cell culture device shown in FIG. 1;

FIG. 6 is a cross-sectional view taken along line A-A of the cell culture device shown in FIG. 5;

FIG. 7 is an exploded perspective view of a cell culture device according to another embodiment of the present invention;

FIG. 8 is an assembled perspective view of the cell culture device shown in FIG. 7; and

FIG. 9 is a view showing another form of a lower body shown in FIG. 7.

### DETAILED DESCRIPTION OF THE EMBODIMENTS

Hereinafter, embodiments of the present invention will be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art.

In the drawings, the shapes and dimensions of components may be exaggerated for clarity, and the same reference numerals will be used throughout to designate the same or like components.

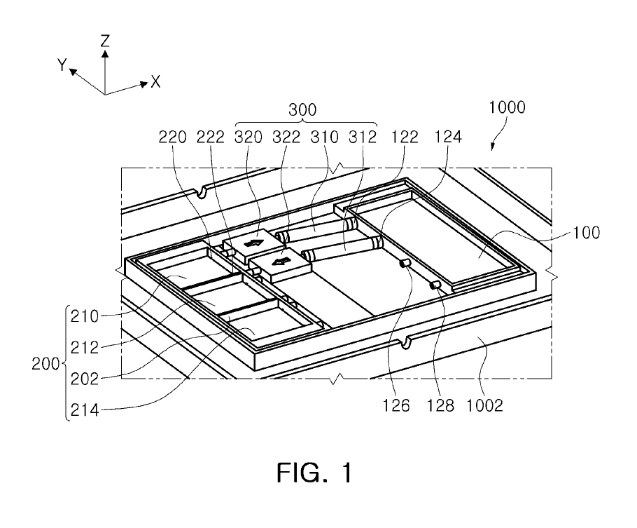

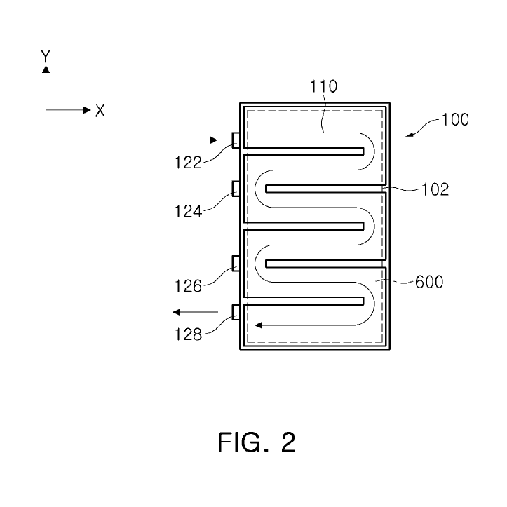

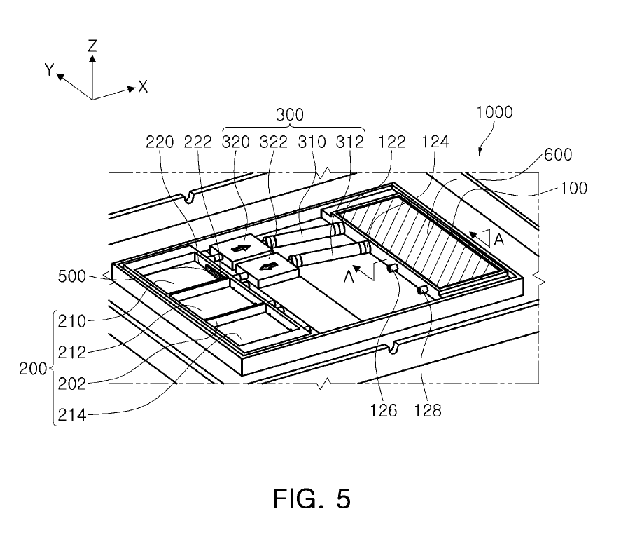

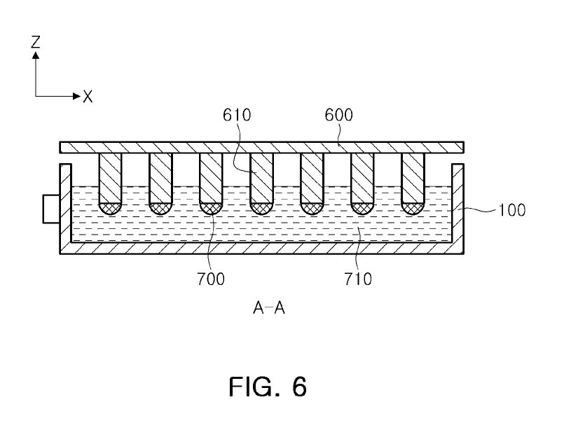

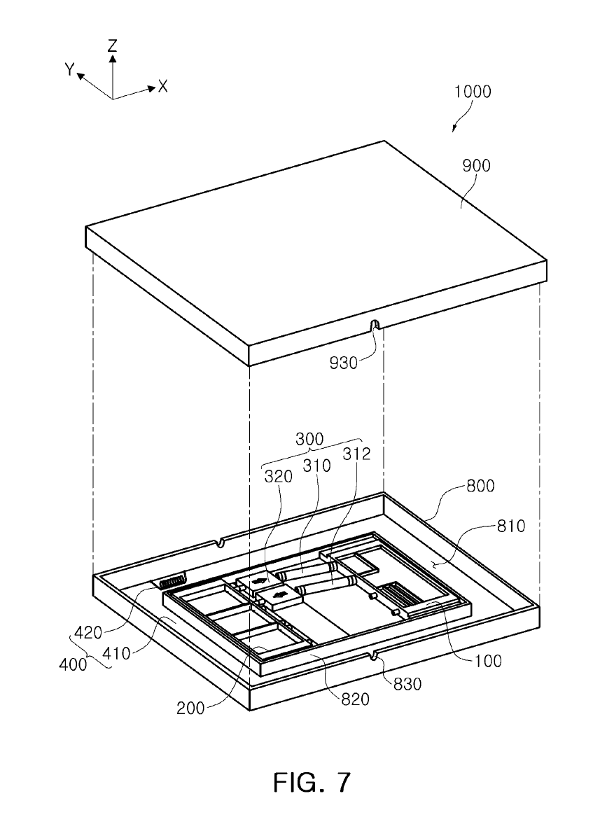

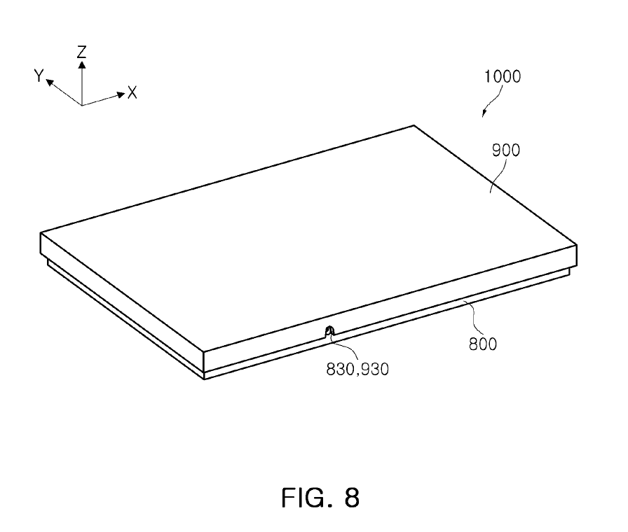

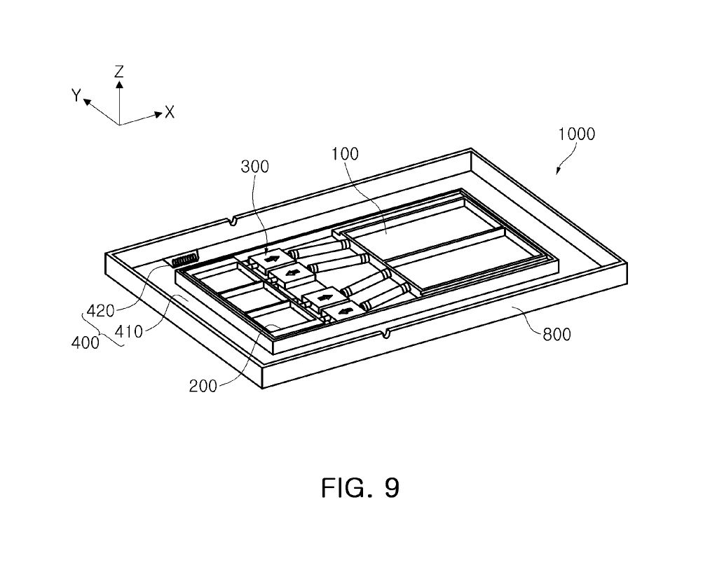

FIG. 1 is a view showing a cell culture device according to an embodiment of the present invention; FIGS. 2 through 4 are plan views showing different forms of a cell chip receiving part shown in FIG. 1; FIG. 5 is a view showing a state in which a cell chip is mounted in the cell culture device shown in FIG. 1; FIG. 6 is a cross-sectional view taken along line A-A of the cell culture device shown in FIG. 5; FIG. 7 is an exploded perspective view of a cell culture device according to another embodiment of the present invention; FIG. 8 is an assembled perspective view of the cell culture device shown in FIG. 7; and FIG. 9 is a view showing another form of a lower body shown in FIG. 7.

A cell culture device 1000 according to an embodiment of the present invention will be described with reference to FIGS. 1 to 6.

The cell culture device 1000 may include a cell chip receiving part 100, a drug storing part 200, and a circulation part 300. Here, the cell chip receiving part 100, the drug storing part 200, and the circulation part 300 may be integrally formed in a single body 1002. Otherwise, the cell chip receiving part 100, the drug storing part 200, and the circulation part 300 may be aggregated into a single body. However, the cell chip receiving part 100, the drug storing part 200, and the circulation part 300 do not have to be integrally formed with the body. For example, any one of the cell chip receiving part 100, the drug storing part 200, and the circulation part 300 may be separated from the body 1002.

The cell chip receiving part 100 may receive at least one cell chip. To this end, the cell chip receiving part 100 may be provided with a space for receiving the cell chip. The cell chip may be mounted in the space in a state in which it is overturned (See FIG. 6).

The cell chip receiving part 100 may include a plurality of first connection tubes 122, 124, 126, and 128 connected to the circulation part 300. The first connection tubes 122, 124, 126, and 128 may be formed in one direction (a Y-axis direction based on FIG. 1) of the cell chip receiving part 100. Here, positions of the first connection tubes 122, 124, 126, and 128 and intervals therebetween are not particularly limited. For example, the intervals between the first connection tubes 122, 124, 126, and 128 may be the same as one another, or may be partially different. In addition, the number of the first connection tubes 122, 124, 126, and 128 is not particularly limited. For example, FIG. 1 shows that the four first connection tubes 122, 124, 126, and 128 are formed in the cell chip receiving part 100; however, the number of the first connection tubes may be increased or decreased as necessary.

Meanwhile, the cell chip receiving part 100 may have a passage 110 inducing the flow (movement) of drug. More specifically, the passage 110 may be formed by a partition 102 partially dividing the cell chip receiving part 100. In the passage 110 as described above, the drugs introduced into the cell chip receiving part 100 may be induced to be sequentially in contact, or react with, at least one kind of bio materials attached to the cell chip.

Various forms of the passage 110 will be described with reference to FIGS. 2 to 4.

According to one form of the passage 110, it may have a zigzag shape by the partitions 102 extended in an X-axis direction as shown in FIG. 2. In this case, the connection tubes 122 and 128 of the cell chip receiving part 100 may be connected to pipes 310 and 312, respectively, and the remaining connection tubes 124 and 126 may be closed. The passage 110 having the above-described form may be appropriate for a case in which different kinds of bio materials are arranged in a Y-axis direction.

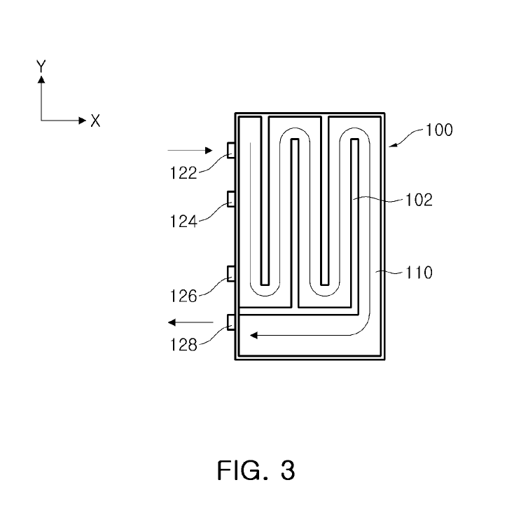

According to another form of the passage 110, it may have a zigzag shape by the partitions 102 extended in a Y-axis direction as shown in FIG. 3. In this case, any one of the connection tubes 122, 124, and 126 of the cell chip receiving part 100 may be connected to the first pipe 310, and the connection tube 128 may be connected to the second pipe 312. This passage 110 may be appropriate for a case in which different kinds of bio materials are arranged in an X-axis direction.

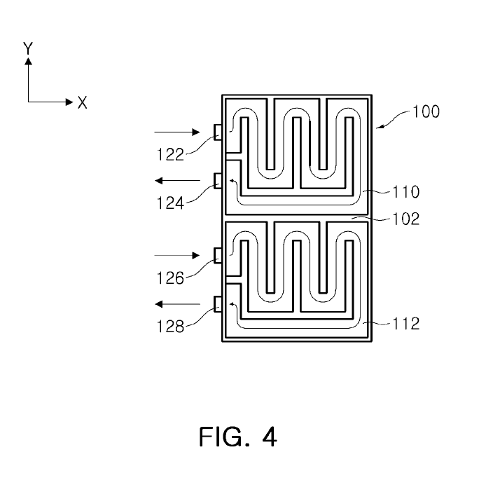

According to another form of passages 110 and 112, the passage may be divided into two regions as shown in FIG. 4. More specifically, one portion of the cell chip receiving part 100 may be provided with a first passage 110 connected from the first connection tube 122 to the first connection tube 124, and the other portion thereof may be provided with a second passage 112 connected from the first connection tube 126 to the first connection tube 128. The passages 110 and 112 as described above may be appropriate for an experiment on different kinds of drug with regard to a single cell chip.

The drug storing part 200 may store the drug. To this end, the drug storing part 200 may include at least one drug storing space 210, 212, and 214. Each of the drug storing spaces 210, 212, and 214 may be divided by partitions 202.

The same kind of drug or different kinds thereof may be stored in the drug storing spaces 210, 212, and 214. As an example, the same kind of drug may be stored in the first drug storing space 210, the second drug storing space 212, and the third drug storing space 214. As another example, different kinds of drugs may be stored in the first drug storing space 210, the second drug storing space 212, and the third drug storing space 214, respectively. As another example, the same kind of drug may be stored in the first drug storing space 210, and the second drug storing space 212, and a different kind of drug may be stored in the third drug storing space 214. However, the forms in which the drugs are stored are not limited to the above-mentioned three examples, but may be varied as necessary. Meanwhile, FIG. 1 shows that the drug storing part 200 is divided into three drug storing spaces 210, 212, and 214; however, the number of drug storing spaces 210, 212, and 214 may be increased or decreased as necessary.

The drug storing part 200 may have a volume equal to or larger than that of the cell chip receiving part 100. For example, the drug storing part 200 may have sufficient volume so that a predetermined amount of drugs are circulated between the cell chip receiving part 100 and the drug storing part 200. Meanwhile, FIG. 1 shows that each volume of the drug storing spaces 210, 212, and 214 is smaller than that of the cell chip receiving part 100; however, each volume of the drug storing spaces 210, 212, and 214 may be equal to or larger than that of the cell chip receiving part 100 as necessary.

The drug storing part 200 may include a plurality of second connection tubes 220 and 222 to be connected to the circulation part 300. The second connection tubes 220 and 222 may be formed in one direction (a Y-axis direction based on FIG. 1) of the drug storing part 200. More specifically, the second connection tubes 220 and 222 may be formed in the drug storing spaces 210, 212, and 214, respectively. Here, the second connection tube 220 may be used as an outlet from which the drug is discharged, and the second connection tube 222 may be used as an inlet into which the discharged drugs are reintroduced. Here, the second connection tubes 220 and 222 are not fixedly used as the outlet and the inlet, respectively, which may be changed as necessary. For example, the second connection tube 222 may be used as the outlet or the second connection tube 220 may be used as the inlet.

The drug storing part 200 may be connected to the cell chip receiving part 100 via the circulation part 300. Therefore, the drug of the drug storing part 200 may be supplied to the cell chip receiving part 100 through the circulation part 300. Similar to this, the drug supplied to the cell chip receiving part 100 may be supplied to the drug storing part 200 through the circulation part 300.

The drug storing part 200 may be disposed to face the cell chip receiving part 100. For example, the drug storing part 200 may be disposed to be symmetrical with regard to the cell chip receiving part 100 based on the Y-axis as shown in FIG. 1. However, the arrangement of the drug storing part 200 is not limited to that shown in FIG. 1, and it may be varied as necessary.

The circulation part 300 may be disposed between the cell chip receiving part 100 and the drug storing part 200. More specifically, the first connection tubes 122, 124, 126, and 128 of the cell chip receiving part 100 and the second connection tubes 220 and 222 of the drug storing part 200 may be connected to one another by the circulation part 300. In addition, the circulation part 300 may allow the drug of the drug storing part 200 to be transferred to the cell chip receiving part 100, and allow the drug transferred to the cell chip receiving part 100 to be introduced to the drug storing part 200. Therefore, the drug of the drug storing part 200 may be circulated between the drug storing part 200 and the cell chip receiving part 100 by the circulation part 300.

The circulation part 300 may include the pipes 310 and 312. More specifically, the first connection tube 122 of the cell chip receiving part 100 and the second connection tube 220 of the drug storing part 200 may be connected to one another by the first pipe 310 and the first connection tube 124 of the cell chip receiving part 100 and the second connection tube 222 of the drug storing part 200 may be connected to one another by the second pipe 312. Meanwhile, these connections are merely exemplary, and may be varied as necessary. For example, the first connection tube 122 of the cell chip receiving part 100 and the second connection tube 220 of the drug storing part 200 may be connected to one another by the first pipe 310 and the first connection tube 128 of the cell chip receiving part 100 and the second connection tube 222 of the drug storing part 200 may be connected to one another by the second pipe 312. In addition, FIG. 1 shows that the cell chip receiving part 100 and the first drug storing space 210 are connected to one another by the pipes 310 and 312; however, the cell chip receiving part 100 may be connected to the second drug storing space 212 or the third drug storing space 214 as necessary.

The circulation part 300 may further include pumps 320 and 322. The pumps 320 and 322 may be mounted on the pipes 310 and 312 or the connection pipes 122, 124, 126, 128, 220, and 222 so that the drug is transferred. For example, the drug of the drug storing part 200 may be transferred to the cell chip receiving part 100 by a first pump 320 mounted on the first pipe 310, and the drug of the cell chip receiving part 100 may be transferred to the drug storing part 200 by a second pump 322 mounted on the second pipe 312. Meanwhile, the pumps 320 and 322 are mounted on the first pipe 310 and the second pipe 312 in the present embodiment, respectively; however, the first pump 320 or the second pump 322 may be omitted as necessary.

Meanwhile, the circulation part 300 may be freely coupled to and separated from the cell chip receiving part 100 and the drug storing part 200. Therefore, any one of the drug storing spaces 210, 212, and 214 may be selected to conduct a drug experiment according to an experiment purpose with regard to the cell chip mounted on the cell chip receiving part 100 by a user.

The cell culture device 1000 as configured above may receive at least one cell chip 600 in the cell chip receiving part 100 as shown in FIG. 5. Here, the cell chip 600 may be disposed in a state in which it is overturned as shown in FIG. 6. Therefore, a bio material 700 adhered to a pillar 610 of the cell chip 600 may be reacted with a drug 710 supplied into the cell chip receiving part 100. Here, since the drug 710 is continuously circulated between the cell chip receiving part 100 and the drug storing part 200 through the circulation part 300, the reaction between the drug 710 and the bio material 700 may be observed in an environment similar to that inside a body, and an experiment and an observation on the bio material 700 with regard to the drug 710 may be conducted for a long period of time.

In addition, the cell culture device 1000 allows for various forms of passages 110 in the cell chip receiving part 100, thereby creating an environment such as in vivo experiment or culture in vivo. Therefore, an experiment and an observation with respect to an effect of the drug 710 on human beings may be relatively accurately conducted in the present cell culture device 1000.

Further, an experiment and an observation of continuous supply of the drug, waste materials accumulation, and the reaction of the bio material 700 for a change in concentration of the drug accordingly may be conducted in the cell culture device 1000.

Meanwhile, the cell culture device 1000 may include a filter 500 mounted in the cell chip receiving part 100 or the drug storing part 200, whereby foreign materials generated during the reaction between the drug 710 and the bio material 700 may be selectively removed. In addition, the foreign materials filtered by the filter 500 are collected, and the foreign materials or waste materials generated during the reaction between the drug 710 and the bio material 700 may be separately observed.

Hereinafter, a cell culture device according to another embodiment of the present invention will be described with reference to FIGS. 7 to 9. For reference, in the present embodiment, the same reference numerals will be used to describe the same components as those of the first embodiment. In addition, a detailed description of these components will be omitted.

The cell culture device 1000 according to the present embodiment may further include a lower body 800 and an upper body 900.

The lower body 800 may be provided with a receiving space 810. The receiving space 810 may be provided with a main wall 820 separating a central portion thereof from an edge portion thereof. Therefore, the receiving space 810 may be divided into two portions by the main wall 820. Here, all of the cell chip receiving part 100, the drug storing part 200, and the circulation part 300 may be received in space (a first space) surrounded by the main wall 820. In addition, an outer portion (a second space) separated by the main wall 820 may be provided with a humidity control part 400.

The humidity control part 400 may include a water storing space 410 and a heater 420. The water storing space 410 may store considerable amount of water, and the heater 420 may heat water stored in the water storing space 410. The humidity control part 400 may generate water vapor around the main wall 820, whereby the humidity inside the cell culture device 1000 may be constantly maintained. Meanwhile, FIGS. 7 and 8 show that the heater 420 is formed in a portion of the water storing space 410; however, it may be formed over the entire area of the water storing space 410 as necessary. In addition, the heater 420 may be omitted.

The upper body 900 may be coupled to the lower body 800. More specifically, the upper body 900 may be coupled to the lower body 800, so that the receiving space 810 is closed. The upper body 900, as configured above, may prevent foreign materials from being introduced into the cell culture device 1000 and protect the cell chip receiving part 100 and the drug storing part 200 from external impacts.

The lower body 800 and the upper body 900 may be provided with holes 830 and 930, respectively. Electric wires (not shown) connecting the pumps 310 and 312 to an external power supply may be led to the holes 830 and 930. However, the holes 830 and 930 are not only used as holes allowing the wires to be led out, but may also be used as air passages as necessary.

As set forth above, a cell culture device according to embodiments of the present invention may allow for the observation or examination of a reaction between a cell and a drug in an environment similar to that of the inside of a body.

Therefore, with the cell culture device according to the embodiments of the present invention, the reliability of experimental results on the reaction between the drug and the cell may be increased.

While the present invention has been shown and described in connection with the embodiments, it will be apparent to those skilled in the art that modifications and variations can be made without departing from the spirit and scope of the invention as defined by the appended claims.

What is claimed is:

1. A cell culture device comprising:
a cell chip receiving part receiving a cell chip;
a drug storing part storing a drug; and
a circulation part connecting the cell chip receiving part and the drug storing part to one another and circulating the drug therebetween.

2. The cell culture device of claim 1, wherein the cell chip receiving part has a passage determining a path for movement of the drug.

3. The cell culture device of claim 2, wherein the passage has a zigzag shape.

4. The cell culture device of claim 1, wherein the drug storing part is divided into a plurality of drug storing spaces so as to individually store the same or different types of drug.

5. The cell culture device of claim 4, wherein the circulation part is coupled to and separated from the cell chip receiving part and the drug storing part so as to selectively connect the plurality of drug storing spaces and the cell chip receiving part to one another.

6. The cell culture device of claim 1, wherein the circulation part includes:
a pair of pipes connecting the drug storing part and the cell chip receiving part to one another; and
a pump installed on the pipe.

7. The cell culture device of claim 1, wherein at least one of the cell chip receiving part and the drug storing part includes a filter for filtering a foreign material included in the drug circulating between the cell chip receiving part and the drug storing part.

8. A cell culture device comprising:
a lower body having a receiving space;
a cell chip receiving part mounted in the receiving space and receiving a cell chip;
a drug storing part mounted in the receiving space and storing a drug;
a circulation part connecting the cell chip receiving part and the drug storing part to one another and circulating the drug therebetween; and
an upper body coupled to the lower body to close the receiving space.

9. The cell culture device of claim 8, wherein the cell chip receiving part has a passage determining a path for movement of the drug.

10. The cell culture device of claim 9, wherein the passage has a zigzag shape.

11. The cell culture device of claim 8, wherein the drug storing part is divided into a plurality of drug storing spaces so as to individually store the same or different types of drug.

12. The cell culture device of claim 11, wherein the circulation part is coupled to and separated from the cell chip receiving part and the drug storing part so as to selectively connect the plurality of drug storing spaces and the cell chip receiving part to one another.

13. The cell culture device of claim 8, wherein the circulation part includes:
a pair of pipes connecting the drug storing part and the cell chip receiving part to one another; and
a pump installed on the pipe.

14. The cell culture device of claim 8, wherein at least one of the cell chip receiving part and the drug storing part includes a filter for filtering a foreign material included in the drug circulating between the cell chip receiving part and the drug storing part.

15. The cell culture device of claim 8, further comprising a humidity control part formed in the receiving space to control humidity therein.

16. The cell culture device of claim 15, wherein the humidity control part includes:
a water storing space storing water; and
a heater heating the water in the water storing space.
